(19) United States
(12) Patent Application Publication
(10) Pub. No.: US 2026/0366006 A1
(43) Pub. Date: Nov. 19, 2026

(54) METHOD OF CONTROLLING GENE EXPRESSION OF CANCER CELL DNA USING TERAHERTZ WAVE

(71) Applicant: UNIVERSITY OF SEOUL INDUSTRY COOPERATION FOUNDATION, Seoul (KR)

(72) Inventor: Joo-Hiuk SON, Seoul (KR)

(73) Assignee: UNIVERSITY OF SEOUL INDUSTRY COOPERATION FOUNDATION, Seoul (KR)

(21) Appl. No.: 19/133,603

(22) PCT Filed: Nov. 23, 2023
(86) PCT No.: PCT/KR2023/019006

§ 371 Date: May 28, 2025

(30) Foreign Application Priority Data
Dec. 9, 2022 (KR) 10-2022-0171224

(51) Int. Cl. C12N 13/00; C12Q 1/6886

(52) U.S. Cl. CPC C12N 13/00; C12Q 1/6886; C12Q 2600/158

(57) ABSTRACT

The present invention relates to a method of controlling gene expression of cancer cells capable of controlling demethylation of cancer cells without damaging DNA. The method of controlling gene expression of cancer cells according to the present invention includes: (a) preparing a control sample cancer cell and a first sample cancer cell to an N^{th} sample cancer cell; (b) irradiating terahertz waves with different powers to the first sample cancer cell to the N^{th} sample cancer cell, respectively; (c) comparing the first sample cancer cell to the N^{th} sample cancer cell irradiated with the terahertz waves with different powers in the step (b) with the control sample cancer cell to detect an expression level of a predetermined gene of each of the first sample cancer cell to the N^{th} sample cancer cell; and (d) irradiating a terahertz wave to a target cancer cell with a power of the terahertz wave irradiated to a sample cancer cell of the first sample cancer cell to the N^{th} sample cancer cell, wherein the sample cancer cell has a predetermined expression level compared to the control sample cancer cell to express a predetermined gene of the target cancer cell at the predetermined expression level (where N is a natural number).

### DESCRIPTION

### Technical Field

The present invention relates to a method of controlling gene expression of cancer cells.

### Background Art

Since cancer cells have the ability to self-renew and differentiate into various cells, research is actively underway to use cancer cells in the treatment of degenerative diseases. While treatment using cancer cells initially garnered great expectations, there are reports of problems in which the regeneration of damaged tissue is not as sufficient as expected or the effect is not sustained. This is because the survival ratio of transplanted adult cancer cells in the body and the ability to differentiate into desired cells are low.

Epigenetic modification is recognized as an important factor in the differentiation of cancer cells. By controlling the gene activity of cancer cells, it is possible to control which cells the cancer cells differentiate into and to what stage the cancer cells will differentiate in the development of embryos and the differentiation of cancer cells. That is, when the gene activity of cancer cells is appropriately controlled, cancer cells may be controlled to differentiate into cells of a desired lineage.

Although many studies have been conducted to control the expression of cancer cells employing biochemical reactions using enzymes, there are problems with low reproducibility and DNA damage.

Therefore, it is necessary to develop a cell therapy agent that maximizes the efficiency of cancer cell treatment through the development of a technology that more precisely controls the differentiation of cancer cells.

### DETAILED DESCRIPTION OF THE INVENTION

### Technical Problem

It is an object of the present invention to provide a method of controlling gene expression of cancer cells.

### Technical Solution

The method of controlling gene expression of cancer cells according to the present invention includes (a) preparing a control sample cancer cell and a first sample cancer cell to an N^{th} sample cancer cell; (b) irradiating terahertz waves with different powers to the first sample cancer cell to the N^{th} sample cancer cell, respectively; (c) comparing the first sample cancer cell to the N^{th} sample cancer cell irradiated with the terahertz waves with different powers in the step (b) with the control sample cancer cell to detect an expression level of a predetermined gene of each of the first sample cancer cell to the N^{th} sample cancer cell; and (d) irradiating a terahertz wave to a target cancer cell with a power of the terahertz wave irradiated to a sample cancer cell of the first sample cancer cell to the N^{th} sample cancer cell, wherein the sample cancer cell has a predetermined expression level compared to the control sample cancer cell to express a predetermined gene of the target cancer cell at the predetermined expression level (where N is a natural number).

The method of controlling gene expression of a cancer cell according to the present invention may further include storing the first sample cancer cells to the N^{th} sample cancer cells for a predetermined period of time after performing step (b).

It is preferable that the predetermined period of time is within 48 hours.

Each of the first sample cancer cells to the N^{th} sample cancer cells may include a melanoma cell.

The target cancer cell may include a melanoma cell.

### Advantageous Effects

The method of controlling gene expression of cancer cells according to the present invention has the following advantages.
(1) The method of controlling gene expression of cancer cells according to the present invention is advantageous in that the expression of a specific gene is induced without damaging DNA and cells. That is, even when terahertz waves are irradiated to cancer cells, AP sites hardly increase such that DNA of cancer cells is not damaged, and thus expression of a specific gene may be induced without damaging DNA of cancer cells.
(2) The method of controlling gene expression of cancer cells according to the present invention is advantageous in that a desired gene expression level is obtained depending on the power of the terahertz wave being irradiated and the sample storage time.

### BRIEF DESCRIPTION OF DRAWINGS

FIG. 1 is a flow chart illustrating a method of controlling gene expression of cancer cells according to the present invention.

FIG. 2 is a schematic diagram illustrating a step S200 of the method of controlling gene expression of cancer cells according to the present invention.

FIG. 3 is a graph illustrating the number of genes over time satisfying FC>2 according to the present invention.

FIGS. 4A to 4C are graphs illustrating gene expression levels of JUN, FOS and CXCL8 according to the method of controlling gene expression of cancer cells according to the present invention, respectively.

### BEST MODE FOR CARRYING OUT THE INVENTION

Hereinafter, a method of controlling gene expression of cancer cells according to the present invention will be described in detail with reference to the attached drawings.

First, the method of controlling gene expression of cancer cells according to the present invention is performed in a terahertz wave irradiation device, a methylation level detection device, etc.

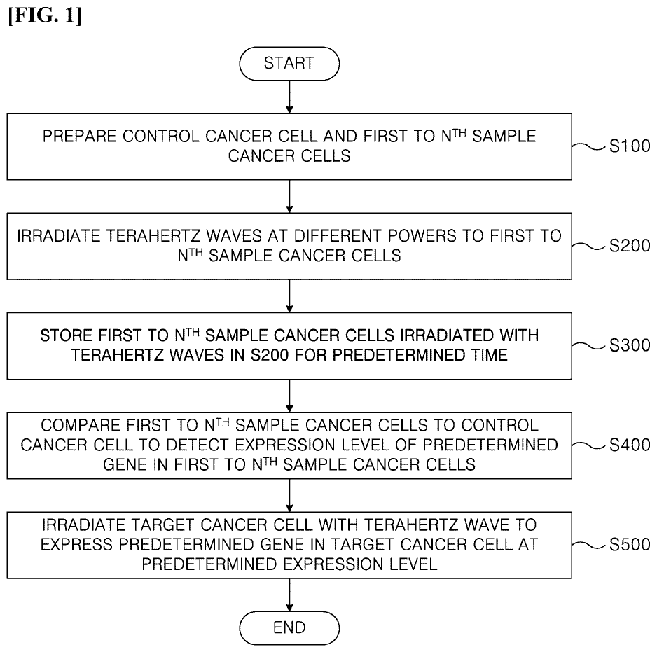

FIG. 1 is a flow chart illustrating the method of controlling gene expression of cancer cells according to the present invention.

Referring to FIG. 1, a control sample cancer cell and a first sample cancer cell to an N^{th} sample cancer cell are prepared (S100) (N is a natural number greater than or equal to 1). The control sample cancer cell and the first sample cancer cell to the N^{th} sample cancer cell are substantially the same cancer cells. According to the present invention, the control sample cancer cell is not exposed to terahertz waves while the first sample cancer cell to the N^{th} sample cancer cell are cancer cells to which terahertz waves are irradiated.

Here, it is preferable that each of the control sample cancer cell and each of the first sample cancer cell to the N^{th} sample cancer cell include melanoma cells.

Next, terahertz waves are irradiated to each of the first sample cancer cell to the N^{th} sample cancer cell (S200).

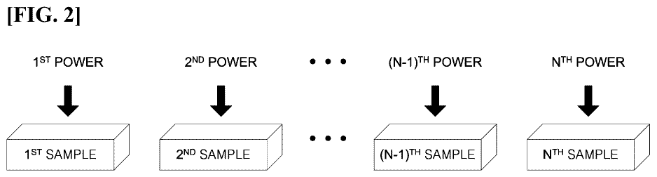

The Step S200 will be described in detail with reference to FIG. 2.

FIG. 2 is a schematic diagram illustrating the step S200 of the method of controlling gene expression of cancer cells according to the present invention.

Referring to FIG. 2, terahertz waves are irradiated to the first sample cancer cell to the N^{th} sample cancer cell at the first power to the N^{th} power, respectively.

Specifically, the first sample cancer cell is irradiated with the terahertz wave of the first power, and the second sample cancer cell is irradiated with the terahertz wave of the second power. Similarly, the (N−1)^{th} sample cancer cell is irradiated with the terahertz wave of the (N−1)^{th} power, and the N^{th} sample cancer cell is irradiated with the terahertz wave of the N^{th} power.

Here, the power of the terahertz wave may be appropriately selected as needed and is not limited to a specific value.

Thereafter, the first sample cancer cell or the N^{th} sample cancer cell irradiated with the terahertz wave in the step S200 is stored until a predetermined time has elapsed (S300).

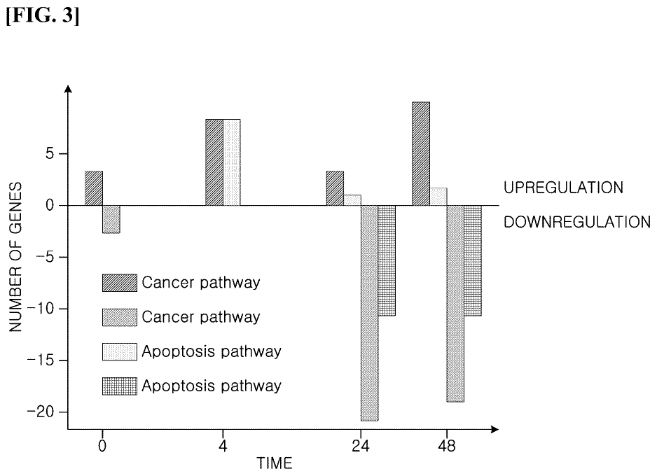

Here, it is preferable that the predetermined time is within 48 hours. The reason for storing the first sample cancer cell or the N^{th} sample cancer cell irradiated with the terahertz wave until a predetermined time has elapsed is that, as shown in FIG. 3, the expression of genes changes over time. This will be described later.

Next, the first sample cancer cell to N^{th} sample cancer cell irradiated with the terahertz wave of first power to N^{th} power in step S200 is compared to the control sample cancer cell after the predetermined time has elapsed in step S300 (S400). When the sample cancer cell is irradiated with the terahertz wave, a change occurs in the expression of the gene of the sample cancer cell. Therefore, in step S400, the expression level of the specific gene of the control sample cancer cell that is not irradiated with the terahertz wave and the expression levels of the specific gene of the first sample cancer cell to N^{th} sample cancer cell that is irradiated with the terahertz wave are compared.

By this comparison, the relative expression level of the specific gene according to the power of the irradiated terahertz wave is detected.

The relative expression level of the gene may be performed by differential gene expression analyses. Specifically, by extracting RNA from a control sample cancer cell and a sample cancer cell irradiated with terahertz waves and performing differential gene expression analysis using Affymetrix GeneChip Affymetrix Human 2.0ST microarray (Affymetrix, Santa Clara, CA, USA), the relative expression level of the gene may be obtained.

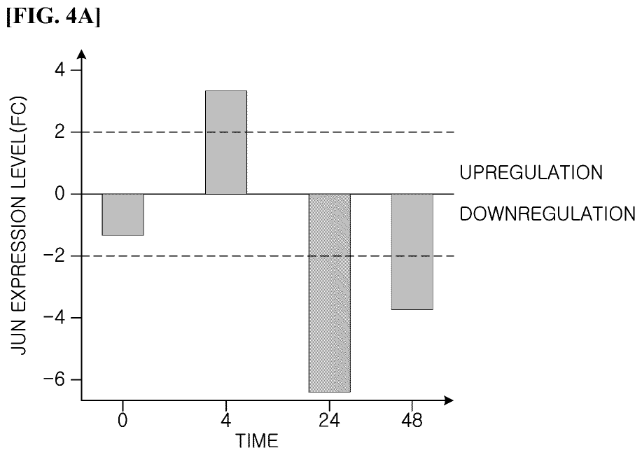

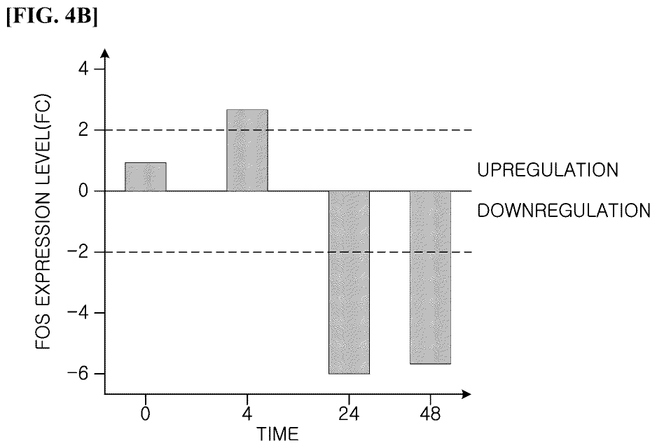

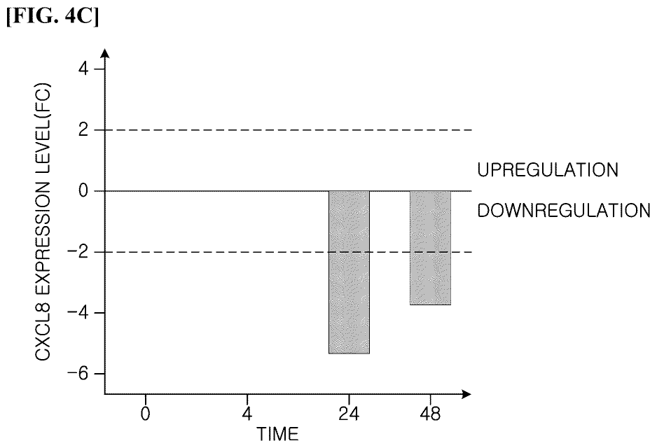

Hereinafter, a Step S400 will be described in detail with reference to FIGS. 3 and 4A to 4C.

When a cancer cell is exposed to a terahertz wave, different gene expressions appear depending on the type of gene of the cancer cell. In addition, the expression level of the gene changes depending on the time elapsed since the cancer cell was exposed to the terahertz wave.

FIG. 3 is a graph illustrating the number of genes over time satisfying FC>2 according to the present invention. That is, the number of genes with a change amount of 2 times or more (2 fold change (FC)). Here, “more than 2 times” means that the expression level of the specific gene of the sample cancer cell irradiated with the terahertz wave is more than 2 times that of the specific gene of the control sample cancer cell. For example, when the FC value of the graph is 5, it means that the expression amount of the gene is 5 times that of the control sample cancer cell.

In order to confirm the effect of the present invention, the expression of about 44,000 genes was analyzed. First, genes with an expression level of 2FC or more among the 44,000 genes were identified. Next, the number of genes affecting the progression to cancer (cancer pathway) and the number of genes that undergo apoptosis among the genes with an expression level of 2FC or more were confirmed.

As shown in FIG. 3, after 24 hours, the number of genes related to the cancer pathway that are upregulated and downregulated are about 3 and 22, respectively, and the number of genes related to natural death are about 1 and 11, respectively. In addition, after 48 hours, the number of genes related to the cancer pathway that are upregulated and downregulated are about 6 and 18, respectively, and the number of genes related to natural death that are upregulated and downregulated are about 2 and 11, respectively. For convenience, the number of upregulated genes is indicated as a positive number and the number of downregulated genes is indicated as a negative number in FIG. 3.

Referring to FIG. 3, it is confirmed that the number of downregulated genes increases significantly as 24 hours pass. Here, upregulation and downregulation mean that specific genes are activated and deactivated by external stimulation, respectively.

FIGS. 4A to 4C are graphs illustrating gene expression levels of JUN, FOS and CXCL8 according to the method of controlling gene expression of cancer cells according to the present invention, respectively.

In particular, FIGS. 4A to 4C illustrate the expression levels of JUN, FOS and CXCL8 genes, which are related to cancer cells among genes having expression levels of 2FC or higher.

Specifically, the JUN gene and FOS gene are genes that encode dimerized proteins to form activator protein (AP-1) transcription factors that regulate cellular processes of differentiation, proliferation and apoptosis. In addition, the CXCL8 gene is a major transcription and inflammatory factor in melanoma progression.

Referring to FIG. 4A, it is seen that the expression level of the JUN gene of the sample cancer cell irradiated with the terahertz wave illustrates a large downregulation after 24 hours. This means that the irradiation with the terahertz wave leads to stimulation of transcription factors and the abnormal overexpression of the gene is gradually readjusted.

Similarly, referring to FIG. 4B and FIG. 4C, it is seen that the FOS gene and CXCL8 gene of the sample cancer cell irradiated with the terahertz wave also illustrate a large downregulation after 24 hours.

Here, when the expression level of the specific gene is higher in the control sample cancer cell, the ratio of the gene expression level of the control sample cancer cell to that of the sample cancer cell is expressed as the FC value. Conversely, when the expression level of the specific gene is higher in the sample cancer cell, the ratio of the gene expression level of the sample cancer cell to that of the control sample cancer cell is expressed as the negative FC value.

Thereafter, the terahertz wave is irradiated to the target cancer cell with the power of the terahertz wave irradiated to the sample cancer cell having the predetermined expression level compared to the control sample cancer cell among the first sample cancer cell to the N^{th} sample cancer cell, thereby causing the predetermined gene of the target cancer cell to be expressed at the predetermined expression level (S500).

Each gene of cancer cells has a different expression level depending on the power of the terahertz wave being irradiated. In particular, the expression level of a specific gene may be controlled depending on the power of the terahertz wave being irradiated and the time elapsed after the terahertz wave is irradiated.

1. A method of controlling gene expression of cancer cells, comprising:
(a) preparing a control sample cancer cell and a first sample cancer cell to an N^{th} sample cancer cell;
(b) irradiating terahertz waves with different powers to the first sample cancer cell to the N^{th} sample cancer cell, respectively;
(c) comparing the first sample cancer cell to the N^{th} sample cancer cell irradiated with the terahertz waves with different powers in the step (b) with the control sample cancer cell to detect an expression level of a predetermined gene of each of the first sample cancer cell to the N^{th} sample cancer cell; and
(d) irradiating a terahertz wave to a target cancer cell with a power of the terahertz wave irradiated to a sample cancer cell of the first sample cancer cell to the N^{th} sample cancer cell, wherein the sample cancer cell has a predetermined expression level compared to the control sample cancer cell to express a predetermined gene of the target cancer cell at the predetermined expression level (where N is a natural number).

2. The method of claim 1, further comprising: storing the first sample cancer cells to the N^{th} sample cancer cells for a predetermined period of time after performing step (b).

3. The method of claim 2, wherein the predetermined period of time is within 48 hours.

4. The method of claim 1, wherein each of the first sample cancer cells to the N^{th} sample cancer cells comprises a melanoma cell.

5. The method of claim 2, wherein each of the first sample cancer cells to the N^{th} sample cancer cells each comprises a melanoma cell.

6. The method of claim 1, wherein the target cancer cell comprises a melanoma cell.
